(19) United States
(12) Patent Application Publication
(10) Pub. No.: US 2025/0121019 A1
(43) Pub. Date: Apr. 17, 2025

(54) METHODS AND SYSTEMS FOR MAKING OIL-LOADED POWDERS AND USE THEREOF

(71) Applicant: MEDISCA PHARMACEUTIQUE INC., Saint-Laurent (CA)

(72) Inventor: Marc JOINER, Scottsdale, AZ (US)

(73) Assignee: MEDISCA PHARMACEUTIQUE INC., Saint-Laurent (CA)

(21) Appl. No.: 18/938,397

(22) Filed: Nov. 6, 2024

Related U.S. Application Data
(63) Continuation of application No. PCT/CA2023/051524, filed on Nov. 14, 2023.
(60) Provisional application No. 63/425,177, filed on Nov. 14, 2022.
(60) Provisional application No. 63/465,590, filed on May 11, 2023.

(51) Int. Cl. A61K 36/185; A61K 8/02; A61K 8/9789; A61K 9/16; A61Q 19/00

(52) U.S. Cl. CPC A61K 36/3482; A61K 8/0279; A61K 8/9789; A61K 9/1694; A61Q 19/00

(57) ABSTRACT

The task of loading oils into carrier particles can present significant technical challenges from an industrial perspective. The solution described herein includes mixing ingredients to produce the oil-loaded powder with a mixer imparting superimposed rotation and revolution movements to a container containing the ingredients, where the ingredients include a carrier in particulate form and an oil, and where the carrier is configured for (i.e., capable of) oil sorption. The oil-loaded powders have a substantially uniform distribution of the oil throughout the particles.

### CROSS-REFERENCE TO RELATED APPLICATIONS

The present application claims the benefit of U.S. provisional patent application Ser. No. 63/425,177 filed on Nov. 14, 2022 and U.S. provisional patent application Ser. No. 63/465,590 filed on May 11, 2023. The contents of each of the above-referenced documents are incorporated herein by reference in their entirety.

### TECHNICAL FIELD

The present disclosure generally relates to the field of methods and systems of making oil-loaded powders, in particular to methods and systems for loading oils into carriers in particulate form to obtain oil-loaded powders, and to processes for incorporating such oil-loaded powders into consumer products and precursors thereof.

### BACKGROUND

Processing natural or synthetic oils for incorporation into consumer products often necessitates converting a liquid or semisolid oil into an oil-loaded free flowing powder by loading carrier particles with the oil. This practice finds widespread utility across various industries, including agrochemical, pharmaceutical, medical, food, textile, cosmetic, and hygiene applications.

The task of loading oils into carrier particles, however, can present significant technical challenges from an industrial perspective. One notable technical challenge arises in cases of oils with higher viscosities, such as plant-extracted oils. Sorption of higher viscosity oils can be time-consuming, higher viscosities requiring extended durations for complete sorption. Attempts to expedite sorption by reducing oil viscosity through means such as the use of solvents or temperature adjustments has been successful but to the detriment of heightened production complexity and cost, which are undesirable from a commercialization perspective.

In light of the aforementioned challenges and limitations, there exists an ongoing need for the development of processes and systems that can produce oil-loaded free-flowing powders while mitigating some of the deficiencies associated with current methods and systems.

### SUMMARY

This Summary is provided to introduce a selection of concepts in a simplified form that are further described below in the Detailed Description. This Summary is not intended to identify key aspects or essential aspects of the claimed subject matter.

In one broad aspect, the present disclosure relates to a method for manufacturing an oil-loaded powder, the method comprising a) placing ingredients in a container body of a container; and b) mixing the ingredients to produce the oil-loaded powder with a mixer imparting superimposed rotation and revolution movements to the container containing the ingredients, wherein the ingredients comprise a carrier in particulate form and an oil, wherein the carrier is configured for oil sorption.

In non-limiting embodiments, the method may include one or more of the following features:
the mixer is a planetary mixer;
the mixer is a bladeless mixer;
the carrier includes micron size particles having a surface area or porous structure capable of oil sorption;
the carrier is a mesoporous particle;
the carrier includes microcrystalline cellulose, silicon dioxide, hydrated colloidal silica, or any combination thereof;
the carrier includes a mesoporous internal network for sorption of the oil;
the mixing includes selecting and/or obtaining a mixing parameter;
the mixing parameter includes mixing time, mixing speed, maximal g force, container size, ingredients amounts, or a combination thereof;
the oil-loaded powder is characterized with a substantially uniform oil distribution and/or substantially uniform mean particle size;
the oil is a plant-based, animal-based, fossil-based, or synthetic oil;
the plant-based oil is a Cannabis oil;
the ingredients having a ratio oil/carrier particles (w/w) of from about 5:1 to about 1:1.

In one broad aspect, the present disclosure relates to a system for manufacturing an oil-loaded powder, the system comprising a) a carrier in particulate form configured for oil sorption; b) a container having a container body for receiving the carrier in particulate form and an oil, and c) a mixer for receiving the container containing the carrier and the oil, wherein the mixer is configured for imparting superimposed rotation and revolution movements to the container for mixing the carrier and the oil to produce the oil-loaded powder.

In non-limiting embodiments, the system may include one or more of the following features:
the mixer is a planetary mixer;
the mixer is a bladeless mixer;
further comprising an adapter for insertion between the container holder and the container, the adapter being configured for securing the container to the container holder;
the carrier includes micron size particles having a surface area or porous structure capable of oil sorption;
the carrier is a mesoporous particle;
the carrier includes microcrystalline cellulose, silicon dioxide, hydrated colloidal silica, or any combination thereof;
the carrier includes a mesoporous internal network for sorption of the oil;
further comprising a user interface for selecting and/or obtaining one or more mixing operating parameters;
the one or more mixing operating parameters includes a mixing time, a mixing speed, or a combination thereof;
the oil-loaded powder is characterized with a substantially uniform oil distribution and/or substantially uniform mean particle size;
the oil is a plant-based, animal-based, fossil-based, or synthetic oil;
the plant-based oil is a Cannabis oil.

In one broad aspect, the present disclosure relates to a method for manufacturing a consumer product containing an oil-loaded powder, the method comprising a) placing a carrier in particulate form configured for oil sorption and an oil in a container body of a container; b) mixing the carrier and the oil to produce the oil-loaded powder with a mixer configured for imparting superimposed rotation and revolution movements to the container containing the ingredients; and c) incorporating the oil-loaded powder in a base product to produce the consumer product.

In non-limiting embodiments, the method may include one or more of the following features:
the consumer product includes an edible consumer product;
the consumer product includes a pharmaceutical product;
the consumer product includes a cosmetic product.

In one broad aspect, the present disclosure relates to an oil-loaded powder comprising carrier particles loaded with the oil by subjecting the powder and oil to superimposed rotation and revolution movements, wherein the powder has a substantially uniform distribution of the oil throughout the particles.

In non-limiting embodiments, the oil-loaded powder may include one or more of the following features:
the carrier particles have a particle size distribution (PSD) of between about 1 μm and about 1000 μm;
the particles have a specific surface area of between about 25 m²/g to about 1000 m²/g;
the particles have an oil sorption capacity of from about 2.0 ml/g to about 4.0 ml/g;
the powder exhibits an oil concentration gradient having ≤3% relative standard deviation (% RSD), when measured at least at the top, middle and bottom layers of the oil-loaded powder;
the powder has an angle of repose of below 40°;
the oil is a plant-based, animal-based, fossil-based, or synthetic oil;
the plant-based oil is a Cannabis oil.

All features of embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF FIGURES

A detailed description of specific embodiments is provided herein below with reference to the accompanying drawings in which:

FIG. 1 is a non-limiting cross-section view of a container including an oil-loaded powder, which is separated in top, middle and bottom sections, in accordance with embodiments of the present disclosure.

FIG. 2 is a non-limiting flow diagram of an illustrative method to obtain the oil-loaded powder of FIG. 1, in accordance with embodiments of the present disclosure.

FIG. 3 is a non-limiting system for manufacturing oil-loaded powders containing a container for receiving ingredients, a carrier in particulate form, and a mixer capable of imparting superimposed rotation and revolution movements to the container containing the ingredients, in accordance with embodiments of the present disclosure.

FIG. 4A is a non-limiting picture of a container containing a Cannabis oil (crude extract) for processing in the system of FIG. 3, in accordance with embodiments of the present disclosure.

FIG. 4B is a non-limiting picture of an oil-loaded powder obtained from processing the Cannabis oil in the container of FIG. 4A with the system of FIG. 3, in accordance with embodiments of the present disclosure.

FIG. 5A is a non-limiting picture of a container including an oil-containing emulsion cream for processing in the system of FIG. 3, in accordance with embodiments of the present disclosure.

FIG. 5B is a non-limiting picture of the container of FIG. 5A with an additional amount of carrier in particulate form on top of the oil-containing emulsion cream of FIG. 5A, in accordance with embodiments of the present disclosure.

FIG. 5C is a non-limiting picture of an oil-loaded powder obtained from processing the ingredients of FIG. 5B, in accordance with embodiments of the present disclosure.

FIG. 6A is a non-limiting picture of a container containing an oil-containing emulsion cream and a carrier in particulate form.

FIG. 6B is a non-limiting picture of the container of FIG. 6A after processing in the system of FIG. 3, with a portion of the cream that is still humid and stuck to the bottom of the container.

FIG. 6C is a non-limiting picture of the oil-loaded powder retrieved from the container of FIG. 6B, which is clumpy and non-uniform in particle size distribution.

FIG. 7 is a non-limiting flow diagram of an illustrative method to manufacture a consumer product by incorporating an oil-loaded powder, in accordance with embodiments of the present disclosure.

In the drawings, embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

Illustrative embodiments of the disclosure will now be more particularly described. The same features are denoted in all figures by the same reference signs. While the making and using of various embodiments of the present invention are discussed in detail below, it should be appreciated that the present invention provides many applicable inventive concepts that can be embodied in a wide variety of specific contexts. Specific embodiments discussed herein are merely illustrative of specific ways to make and use the disclosure and do not delimit the scope of the disclosure.

The present inventor has developed a method and system to manufacture an oil-loaded powder, which addresses at least some of the above-identified shortcomings of existing methods and systems.

In some embodiments, the method includes mixing ingredients to produce the oil-loaded powder with a mixer imparting movements to a container containing the ingredients, where the ingredients include a carrier in particulate form and an oil, and where the carrier is configured for (i.e., capable of) oil sorption. For example, oil adsorption.

In some embodiments, the carrier particles are solid or semi-solid particles.

In some embodiments, the system includes components to produce the oil-loaded powder. For example, the system may include the carrier in particulate form configured for (i.e., capable of) oil sorption, a container having a container body for receiving an oil and the carrier particles, and a mixer for receiving the container containing the oil and the carrier, and for mixing the oil and the carrier to produce the oil-loaded powder, where the mixer imparts movements to the container containing the oil and the carrier.

In some embodiments, the mixer can be characterized as a planetary mixer; as a mixer configured for imparting superimposed rotation and revolution to the container containing the ingredients; as a bladeless mixer; or as any combination thereof.

In some embodiments, the system and method described herein streamline the manufacturing of oil-loaded powders and advantageously generate oil-loaded powders with substantially uniform distribution characteristics. In some applications, particularly for food, pharmaceutical, or cosmetic applications, such substantially uniform distribution characteristics can be advantageous for achieving consistency in potency and/or dosing. Advantageously, the system and method herein described perform the mixing without addition of external heating.

Without being limited to any particular theory, the present inventor believes that the mixing herein described is sufficiently intense to obtain oil-loaded powders with substantially uniform distribution characteristics in a reduced amount of processing time while being sufficiently gentle to prevent the internal temperature of ingredients being mixed from reaching or getting close to a critical temperature. For example, a temperature where an ingredient would undergo an undesirable phase transition (e.g., evaporate, sublimate), would suffer from phase separation, or would degrade.

The method and system described herein afford one or more advantageous technical characteristics, which will be recognized by the skilled reader in view of the present disclosure. For example, the method and system described herein require a single mixer. Reliance on a single mixer may reduce the number of processing steps and potential sources of cross-contamination, which may otherwise occur with conventional approaches. For example, the method and system described herein do not require conventional atomization equipment or solvents, which are typically used when manufacturing oil-loaded powders, thus reducing manufacturing costs and processing time. For example, the mixing described herein can be implemented in a mixer capable of performing movements on a container containing the ingredients, without mixing blades, which results in less cleaning procedures and may reduce cross-contamination risks.

### System and Method

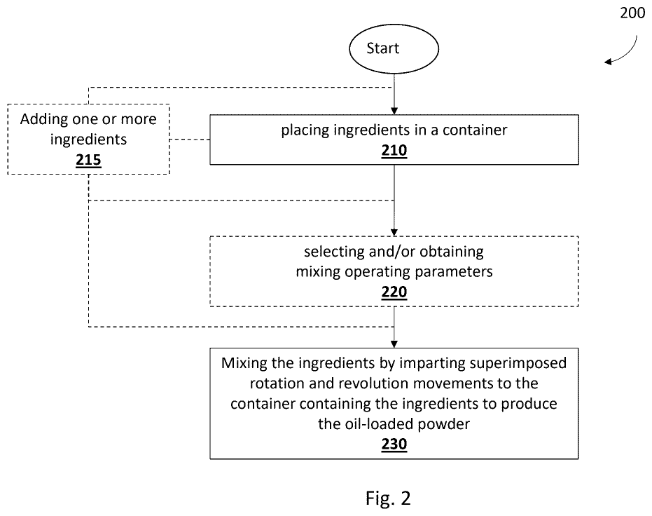

FIG. 2 is a flow chart of a general method 200 of manufacturing an oil-loaded powder, in accordance with an embodiment of the present disclosure.

At step 210, the method 200 includes placing ingredients in a container having a container body for receiving the ingredients. The ingredients may include one or more oil. The ingredients may further include a carrier in particulate form (i.e., carrier particles), where the carrier is configured for oil sorption. For example, oil adsorption. In the context of the present disclosure, the term “oil” refers to natural or synthetic oils, as will be discussed later in this text. The reader will readily understand that the oil may be an isolated oil or an oil-containing composition, which may include, but without being limited to, a cosmetic carrier (e.g., cream, gel, etc.) containing the oil, an emulsion containing the oil, a plant extract containing the oil, and the like. For example, the oil-containing composition may include commercial products such as any one of VersaPro™ Gel, HRT™ Cream, OleaBase™ Plasticized, PLO Gel Mediflo™, Oral Mix™, and VersaPro™ cream, all from Medisca Pharmaceutique (Canada). The carrier in particulate form is also discussed later in this text. The reader will readily understand that various amounts of oil and carrier in particulate form may be used depending on specifics of an application. For example, the oil and carrier in particulate form placed in the container may be in respective amounts such as to obtain a ratio oil/carrier particles (w/w) of from about 5:1 to about 1:1, including any ratio there between, such as about 2:1.

Optionally, at step 215, one or more ingredients can be added to the container before, concomitantly, or after step 210. For example, the one or more ingredients can provide a benefit to a user. Non-limiting examples of the one or more ingredients are described later in this text.

Optionally, at step 220, the method 200 may further include obtaining user input with respect to one or more mixing operating parameters to obtain the desired oil-loaded powder. The user input may involve presenting via a user interface, an operating trigger for implementing the herein described method. Non-limiting examples of such operating trigger may include “encapsulating oil”, “loading carrier with oil”, “powderizing oil”, or any other suitable operating trigger. Alternatively, the user input may involve selecting and/or obtaining via the user interface, the one or more mixing operating parameters to obtain the desired oil-loaded powder. The user interface may be any suitable user interface, such as anyone of those described in U.S. Pat. Nos. 11,338,254, 11,130,106, or WO 2022/217352, all of which are herein incorporated by reference in their entirety.

In some embodiments, the one or more mixing operating parameters can include a mixing time, mixing speed (e.g., one or more of revolution speed, rotation speed, etc.), maximal g force, container size, ingredients amounts (e.g., volume or mass), etc.

In some embodiments, the one or more mixing operating parameters at step 220 may be selected and/or obtained from a set of pre-determined operating parameters.

In some embodiments, the one or more mixing operating parameter includes a mixing time. For example, a mixing time of no more than 990 seconds. For example, the herein described movements imparted on the container may be performed during from about 10 seconds to about 990 seconds, including any suitable value therein. For example, about 10, 15, 20, 25, 30, 35, 40, 45, 50, 55, 60, 75, 100, 120, 150, 180, 240, 300, 400, 500, 600, 700, 800, or 990 seconds.

In some embodiments, the one or more mixing operating parameter includes a maximal G force. For example, a maximal G force of at least 50 g. For example, the herein described movements imparted on the container may be performed with a maximal G force value of less than 500 g, or in the range of 50 g to 400 g, or 75 g to 350 g, or any suitable value within these ranges.

In some embodiments, the one or more mixing operating parameter includes a revolution speed imparted on the container. For example, the revolution speed may be selected from a set of speed levels. For example, a revolution speed of up to about 4000 rpm (revolution per minute). For example, a revolution speed of from about 400 to about 2500 rpm, or any suitable value within these ranges.

In some embodiments, the one or more mixing operating parameter includes a rotation speed imparted on the container. For example, the rotation speed may be selected from a set of speed levels. For example, the rotation speed may be selected from a rotation speed of up to 1000 rpm (rotation per minute). For example, a rotation speed of from about 50 rpm to about 800 rpm.

In some embodiments, the one or more mixing operating parameter includes a revolution:rotation rpm ratio. For example, the revolution:rotation rpm ratio may be selected from a set of revolution:rotation rpm ratios. For example, the revolution:rotation rpm ratio may be of about 10:4.

In some embodiments, the one or more mixing operating parameter discussed above may be individually selectable or may be selectable from pre-determined combinations of parameter values. For example, the ratio between rotation rpm and revolution rpm may be a pre-determined ratio that constrains the revolution rpm for a certain rotation rpm, and vice versa.

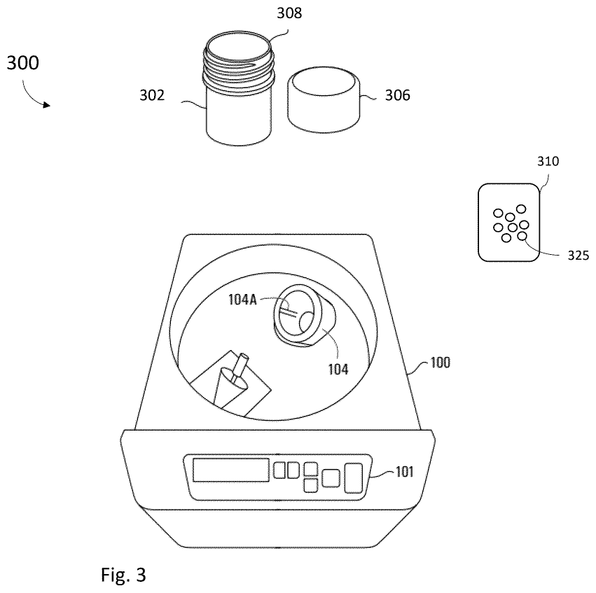

At step 230, the method 200 includes mixing the ingredients to produce the oil-loaded powder by using a mixer that imparts movements to the container containing the ingredients, which are sufficient to produce the oil-loaded powder. The mixing operations can be performed with a system 300, as shown in FIG. 3.

In some embodiments, the system 300 includes a mixer 100, which is capable of performing the desired movements on the container containing the ingredients. For example, the movements may include superimposed revolution and rotational movements. Such movements may be performed on a container holder 104, for example. In some embodiments, the mixer 100 may further include a user interface 101 configured for receiving user input, such as selecting/obtaining mixing operating parameters for performing the herein described movements on the container containing the ingredients. The user interface 101 may be a graphical user interface, for example.

In some embodiments, the system 300 may further include container 302 configured for containing the ingredients. Optionally, the container 302 is also configured for insertion directly into the holder 104. The container may have any suitable shape or size.

In some embodiments, the system 300 may further include carrier particulate form 325 for adding into the container 302 together with an oil. The carrier particulate form 325 can be provided into a container 310, or into any other suitable packaging.

In some embodiments, a commercial package may contain one or more of the mixer 100, the container 302, and the carrier particulate form 325. In some embodiments, the commercial package may include only the mixer 100, where the container 302 and the carrier particulate form 325 are sold separately as consumables.

In some embodiments, the mixer 100 can be a stand-alone device, whereby to use the mixer 100, an operator places the ingredients into the container 302, places the container 302 into the mixer 100, closes a lid thereof (not shown), selects/obtains one or more mixing operating parameters, and pushes start. A suitable non-limiting example may be any of the Maz™ planetary mixer models KK-300SS, KK-400W and KK-1000W, sold by Medisca Pharmaceutique Inc., Canada. The mixer 100 can be communication-enabled or network-enabled of the type described in U.S. Pat. No. 11,130,106 (the content of which are hereby incorporated by reference in its entirety), for communicating with a remote database containing operating parameters for controlling the mixer 100. In some embodiments, the mixer 100 may be part of an automated or semi-automated production cell, in which one or more mixer 100 is (are) incorporated into production lines for downstream applications for manufacturing consumer products including the oil-loaded powders.

In some embodiments, the container 302 and the container holder 104 may be configured for performing solidary superimposed revolution and rotation movements. For example, the holder 104 may include an engaging element for engaging with a corresponding engaging element present on the container to prevent free-spinning of the container within the jar holder 104. As a result, rotation of the container will only occur when the holder 104 holder itself rotates. Other rotational stoppage mechanisms may be provided in different embodiments.

In some embodiments, the container 302 may have an open top end 308. In some embodiment, the container 302 may include open top end 308 and a cover 306. The mouth 308 and the cover 306 may be complementarily threaded.

In some embodiments, the container 302 does not correspond in size (e.g., in volume) to the holder 104. In such embodiments, the system 300 may further include one or more adapter (not shown) for securing the container 302 to the holder 104 such that the superimposed revolution and rotation movements imparted on the holder 104 results in solidary movements of the holder 104, adapter and container 302. Examples of different type of adapters are described for example in U.S. Pat. Nos. 10,993,876, 6,755,565 and 11,478,763, the entire content of each being incorporated by reference herein in their entirety.

In some embodiments, the system 300 may further include an intermediate container (not shown) where the intermediate container has a corresponding volume to that one of the holder 104, such that the intermediate container fits more snuggly within the holder 104. The system 300 may further include an adapter for securing the container 302 within the intermediate container, such that the superimposed revolution and rotation movements imparted on the holder 104 results in solidary movements of the holder 104, the intermediate container, adapter and container 302.

Those ordinarily skilled in the art will appreciate that further improvements may be made to the design of the adapter. In particular, in the case where an adapter is designed that has a maximum width that is less than the width of the holder, rotational motion of the holder may induce slippage in the containing system (which includes the adapter and the container). The amount of slippage may further be a function of the dimensions of the container and the weight and/or volume of the composition contained therein. To reduce slippage, various possible anti-slippage mechanisms may be provided, depending on operational requirements. Non-limiting examples of anti-slippage mechanisms are provided in U.S. Pat. No. 10,993,876, the entire contents of which are hereby incorporated by reference in its entirety.

Without being bound by any theory, it is believed that the superimposed rotation and revolution of the container containing the described ingredients enhance the efficiency of oil loading into the powder particles, leading to the favorable powder characteristics outlined in this text. This dual-motion system is thought to promote the interaction between oil and powder particles, resulting in forces that positively induce the loading of oil into the powder particles.

### Oil-Loaded Powder

In a broad non-limiting aspect, the oil-loaded powders of the present disclosure include carrier particles loaded with oil.

In some embodiments, the oil-loaded powder described herein is a free-flowing powder. In other words, the oil-loaded powder particles are not cohesive—i.e., If particles are cohesive, they cling to one another to form aggregates. The oil-loaded powder described herein may have minimal (e.g., less than about 1 wt. %) or may be free from aggregates.

In some embodiments, the powder particles may have a substantially spherical shape.

In some embodiments, the powder particles may have a mean average micron size of from about 1 micron to about 1000 micron, including any value therein or ranges there in between.

In some embodiments, the oil-loaded powder may further includes one or more ingredient, which is loaded into the carrier particles. For example, the one or more ingredient can include an active pharmaceutical ingredient (API), a cosmetic ingredient, a Cannabis-derived ingredient, etc.

In some embodiments, the oil-loaded powder of the present disclosure includes composite particles (e.g., particles loaded with the oil and the one or more ingredient). In some embodiments, the oil-loaded powder of the present disclosure includes heterogeneous particles, i.e., separate particles of different chemical constitution (e.g., separate particles with the one or more ingredient and separate oil-loaded particles). In some embodiments, the oil-loaded powder of the present disclosure includes a combination of composite particles and heterogeneous particles.

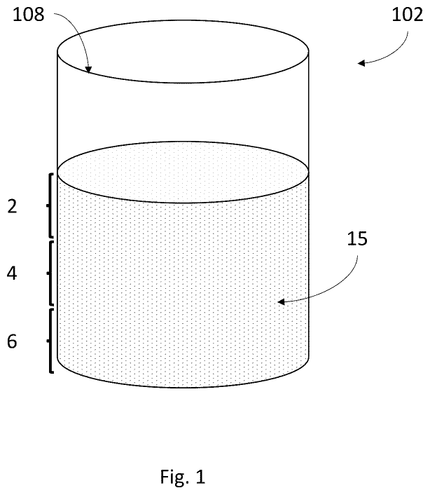

In some embodiments, the oil-loaded powder of the present disclosure is characterized with a substantially uniform (i.e., homogeneous) distribution characteristic. For example, substantially uniform (i.e., homogeneous) oil distribution and/or substantially uniform (i.e., homogeneous) mean particle size distribution. Such uniform (i.e., homogeneous) distribution characteristic can be determined in a top layer, a middle layer and a bottom layer of the oil-loaded powder, for example contained in a container. The substantially uniform (i.e., homogeneous) distribution characteristic can include substantially uniform (i.e., homogeneous) oil distribution and/or mean particle size distribution. The distribution characteristic can be measured using a suitable technique, such as laser light scattering for the particle size distribution or high pressure liquid chromatography (HPLC) for the oil distribution. The standard deviation (SD) between the mean particle size or the oil distribution of the top layer, middle layer and bottom layer can be determined. The relative standard deviation (% RSD), which expresses the precision and repeatability of an assay, can be calculated based on the ratio of the standard deviation to the mean. When the substantially uniform characteristic is determined from a powder contained in a container, this can be determined as illustrated in FIG. 1, which shows a cross-sectional view of a container 102 (which may be the same as container 302) including an oil-loaded powder 15, where the container 102 is virtually separated in top, middle and bottom sections, each including respective top 2, middle 4 and bottom 6 layers of the oil-loaded powder 15.

In some embodiments, the oil-loaded powder exhibits a mean particle size distribution gradient having≤3% relative standard deviation (% RSD), or ≤2% RSD, or ≤1% RSD, or ≤ 0.1% RSD when measured at least at the top 2, middle 4 and bottom 6 layers of the oil-loaded powder. In some embodiments, the mean particle size gradient is about 0% RSD, when measured at least at the top 2, middle 4 and bottom 6 layers of the oil-loaded powder. In some embodiments, the oil-loaded powder exhibits an oil concentration gradient having≤3% relative standard deviation (% RSD), or ≤2% RSD, or ≤1% RSD, or ≤0.1% RSD when measured at least at the top 2, middle 4 and bottom 6 layers of the oil-loaded powder. In some embodiments, the oil concentration gradient is about 0% RSD, when measured at least at the top 2, middle 4 and bottom 6 layers of the oil-loaded powder. As discussed above, the % RSD for the oil distribution can be determined using high pressure liquid chromatography (HPLC)—e.g., when assessing Cannabis oil distribution, the distribution of a cannabinoid (e.g., CBD, THC, etc.) throughout the powder particles can be used.

In some embodiments, the oil-loaded powders of the present disclosure are in a dry form. Within the context of the present disclosure, a “dry oil-loaded powder form” means that the oil-loaded powder has a water activity (a_{w}) of less than 0.75, for example 0.04<a_{w}<0.75, or for example 0.04<a_{w}<0.3. Water activity may be measured using an Aqualab Water Activity Meter 4TE (Decagon Devices, Inc., U.S.A.).

In some embodiments, the oil-loaded powder can be a dispersible oil-loaded powder. Within the context of the present disclosure, a “dispersible oil-loaded powder” means that the oil-loaded powder particles disintegrate when they are contacted with an aqueous solution, thus releasing the oil.

In some embodiments, the oil-loaded powders described herein can have taste barrier properties, which may be useful for various downstream applications in several industries, such as in the pharmaceutical, food, or Cannabis industry. In the Cannabis industry, for example, Cannabis oil-loaded powders can be used for manufacturing consumer edible products that are characterized with an advantageous taste masking property to reduce the edible product's bitterness and off tasting that would otherwise affect the edible product. Such taste-barrier characteristics of oil-loaded powders are known in the art and are further described, for example, in WO 2022/140849.

The fluidity and dispersibility of the oil-loaded powders described herein can be assessed in the context of unconfined solid piles using the angle of repose as an indicator of the oil-loaded powders fluidity. The angle of repose is the angle formed between a horizontal plane and the slope line extending along the face of a pile of material. An angle of repose that is below 40° is believed to be advantageous. Consistency in flowing characteristics is also important when manufacturing solid dosage forms with the herein described oil-loaded powders—i.e., such powders when fed into tableting machines need to flow in exactly the same way from batch to batch. At an angle of repose of more than 40°, the oil-loaded powders are believed to become too cohesive for proper handling and/or result in non-homogeneous tableting mixture which results in poor content uniformity of the active ingredient in the final product. Preferably, the oil-loaded powders have an angle of repose below 36°, preferably below 34°, most preferably below 32°, such as about 30°, measured by a pre-specified analytical method, e.g. determined with a powder flow analyzer to measure the flow behavior of granules and powders in compliance with set standards, such as the EP<2.9.36>, EP<2.9.16>, USP <1174> Pharmacopoeia and ISO 4324 standards. For example, using the Pharmatest PTG-S5 powder flow analyzer.

### Carrier in Particulate Form

In a broad non-limiting aspect, the oil-loaded powders of the present disclosure include carrier particles loaded with oil. Advantageously, the carrier particles have a surface area or porous structure configured for (i.e., capable of) oil sorption. For example, oil adsorption.

In some embodiments, the carrier particles are inert—that is, the carrier particles are chemically non-reactive and solid. The carrier particles may be further characterized in several ways. The carrier particles may be characterized by water solubility (soluble vs. insoluble), by surface area, by structure characteristics (e.g., presence of pores, presence of mesoporous internal network), by application (food, pharmaceutical, cosmetic, Cannabis, etc.), and/or by class (i.e., by the chemical composition of the carrier particles, or the compound from which the carrier particles are derived, etc.).

In some embodiments, the carrier particles may have a mesoporous internal network. As used herein, the term “mesoporous internal network” refers to the presence of internal cavities/pores within the particles of the carrier. For example, the internal cavities/pores can have a diameter for example of between about 2 nm and about 1 μm, including any amount therein between or any ranges therein, according to IUPAC nomenclature.

In some embodiments, the carrier particles are water-soluble. For example, the water-soluble carrier particles may be food-grade carrier particles such as a sugar derivative, starch derivative, fiber derivative, etc. For example, the water-soluble carrier particles can be but without being limited to Fibersol®-2AG (ADM/Matsutani LLC), Promitor® (Tate & Lyle PLC), Orafti® (Beneo GmbH), Star-Dri® 100 (Tate & Lyle PLC), FlowLac® 100 (Meggle), CAPSUL® or CAPSUL® TA (Ingredion Incorporated), N-Lok® (Ingredion Incorporated), N-Zorbit™ M (Ingredion Incorporated), and the like. For example, the water-soluble carrier particles may be pharmaceutical carrier particles, where the chemical composition of the carrier particles is hydroxypropyl cellulose (HPC). For example, the carrier particles can be HPC SSL grade or M grade.

In some embodiment, the carrier particles are water-insoluble carrier particles. For example, the water-insoluble carrier particles can be a food-grade carrier particles such as a starch derivative, fiber derivative, etc. For example, the water-insoluble carrier particles can be but without being limited to N-Zorbit™ 2144 (Ingredion Incorporated), HI-CAP® 100 (Ingredion Incorporated), Mira-Mist® (Tate & Lyle PLC), superior potato starch (KMC), Citri-Fi 100M40 (Fiberstar Inc), and the like. For example, the water-insoluble carrier particles can be an insoluble pharmaceutical carrier particles, where the chemical composition of the carrier comprises cyclodextrin, tricalcium phosphate, carboxymethylcellulose sodium, sodium starch gluconate, croscarmellose sodium, silicon dioxide and the likes, as further described below. For example, the carrier particles can be but without being limited to spray-dried tricalcium phosphate TRI-CAFOS® (Chemische Fabrik Budenheim KG), Tabulose® (Roquette Freres SA), Explosol® (Roquette Freres SA), Solutab® (Roquette Freres SA), spray-dried granular silicon dioxide Fujisil™ (Fuji Chemical Industries USA, Inc), Omyapharm® (Omya International AG), Floguard (PPG Industries. Inc), and the like.

In one embodiment, the carrier particles are a modified starch or starch derivative. As used herein, the term “modified starch” refers to a native starch that has been subjected to a physical, enzymatic and/or chemical treatment to alter at least one of its physicochemical properties. Non limiting examples of modified starches include dextrin (INS 1400), alkaline-modified starch (INS 1402), bleached starch (INS 1403), oxidized starch (INS 1404, E1404), enzyme-treated starch (INS 1405), monostarch phosphate (INS 1410, E1410), distarch phosphate (INS 1412, E1412), acetylated starch (INS 1420, E1420), hydroxypropylated starch (INS 1440, E1440), hydroxyethyl starch, starch sodium octenyl succinate (OSA) starch (INS 1450, E1450), starch aluminium octenyl Succinate (INS 1452, E1452), cationic starch, carboxymethylated starch, phosphated distarch phosphate (INS 1413, E1413), acetylated distarch phosphate (INS 1414, E1414), acetylated distarch adipate (INS 1422, E1422), hydroxypropyl distarch phosphate (INS 1442, E1442), acetylated oxidized starch (INS 1451, E1451) and the likes. The native starch may come from any suitable source, such as a plant or synthetic source. The starch derivative may be maltodextrin.

In some embodiments, the carrier particles include microcrystalline cellulose, silicate-based adsorbent carrier, and colloidal silicon dioxide, or a mixture thereof. For example, a particulate agglomerate of coprocessed microcrystalline cellulose, from about 0.5% to about 50% silicate-based adsorbent carrier, by weight of the microcrystalline cellulose, and optionally from about 0.1% to about 20% colloidal silicon dioxide, by weight. For example, present in a ratio of about 45:45:10. The reader will readily understand that this ratio can be adapted based on the nature of the oil being loaded. For instance, the carrier may include Prosolv® SMCC 50 (silicified microcrystalline cellulose) commercially available from JRS Pharma. For instance, the silicate-based adsorbent carrier is a granulated hydrophilic fumed silica. In certain preferred embodiments, the granulated hydrophilic fumed silica is, e.g., Aeroperl® 300, commercially available from Evonik. For instance, the silicate-based adsorbent carrier is a magnesium aluminometasilicate. In certain preferred embodiments, the magnesium aluminometasilicate is, e.g., Neusilin® US2 or UFL2.

In some embodiments, the carrier particles include microcrystalline cellulose, silicon dioxide, hydrated colloidal silica, magnesium aluminium silicates (MAS), granulated fumed silica (GFS), mesoporous silica gel (MSG), a clay, a starch derivative, or any combination thereof.

In some embodiments, the carrier particles include microcrystalline cellulose, silicon dioxide (silica), hydrated colloidal silica, or any combination thereof.

In some embodiments, the carrier particles include NanoSil™ (Pete pharmaceuticals, USA).

In some embodiments, the carrier particles form can be characterized by a particle size distribution (PSD). For example, the carrier particles have a PSD of between about 1 μm and about 1000 μm, including any amount there between or any ranges therein, in some cases between about 1 μm and about 500 μm, including any amount there between or any ranges therein, about 10 μm and about 250 μm, including any amount there between or any ranges therein. For example, a PSD of about 30 μm, 40 μm, 50 μm, or 60 μm. Particle size can be measured using techniques known in the art, such as with the Laser diffraction technology from Malvern®, Mastersizer® 2000.

In some embodiments, the carrier particles can be characterized as having an average internal cavity/pore volume between about 0.5 cm³/g and about 10 cm³/g, including any amount there between or any ranges therein. For example, the average internal cavity/pore volume of the carrier in some cases can be between about 1 cm³/g and about 7.5 cm³/g, including any amount there between or any ranges therein, in some cases between about 2 cm³/g and about 5 cm³/g, including any amount there between or any ranges therein.

In some embodiments, the carrier particles can be characterized as having a specific surface area of between about 25 m²/g to about 1000 m²/g, including any amount there between or any ranges therein. For example, between about 100 m²/g and about 750 m²/g, between about 200 m²/g and about 500 m²/g, or between about 300 m²/g and about 400 m²/g, including any amount there between or any ranges therein.

In some embodiments, the carrier particles can be characterized as having an oil sorption capacity of from about 2.0 ml/g to about 4.0 ml/g, such as about 2.0 ml/g, about 2.5 ml/g, about 3.0 ml/g, about 3.5 ml/g, or about 4.0 ml/g, and the like. For example, oil adsorption.

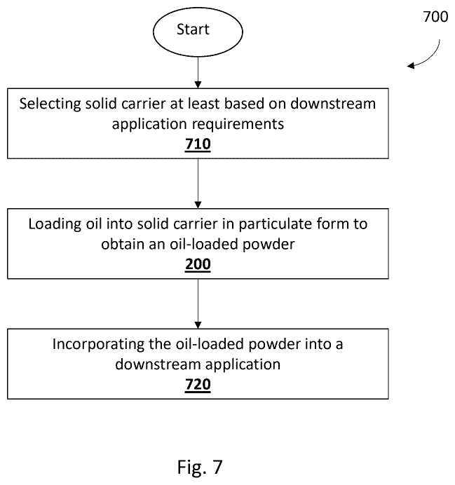

The reader will readily appreciate that there are several carrier particles options for use in the herein described system and method. As illustrate in the flow chart method 700 shown in FIG. 7, one may select carrier particles at least based on downstream application requirements, at step 710. In some embodiments, the downstream application requirements may be prioritized. For example, water-solubility may be a prioritized requirement for incorporating Cannabis oil-loaded powders into Cannabis-infused beverages. Once selected, the user may load oil into the selected carrier particles with the herein described mixing method 200, as shown for example in FIG. 2. At step 720, once the oil-loaded powder is obtained, the user may incorporate such oil-loaded powder into a downstream application to produce a product precursor, or a desired consumer product, such as a Cannabis edible (e.g., foodstuff or beverage), a cosmetic cream, etc.

### Oils

In a broad non-limiting aspect, the oil-loaded powders of the present disclosure include carrier particles loaded with oil.

In some embodiments, the terms “oil” relates to any naturally occurring or synthetically occurring oil. It also includes blends or mixtures of one or more naturally occurring and/or synthetically occurring oils.

Naturally occurring oils include plant-based, animal-based, and fossil-based oils.

Examples of plant-based oils include plant fats and oils extracted from plant or seeds thereof. These may include edible oils (e.g., avocado oil, olive oil, etc.), essential oils (e.g., peppermint, spearmint, etc.), multipurpose oils (e.g., coconut oil, palm oil, etc.), and the like. Plant-based oils may include candelilla oil, ouricury oil, jojoba plant oil, bayberry oil, Japan oil, sunflower oil, tall oil, tallow oil, rice oil, tallows, etc. Other plant-based oils include Cannabis oils, such as full spectrum extracts, crude Cannabis oil, or purified cannabinoid oils, which are described later in this text.

Examples of animal-based oil includes fatty acids such as caprylic, lauric, myristic, oleic, palmitic, and stearic acids, cerebrosides, glycerin, cholesterol, emu oil, fish oil, lanolin, and the like.

Examples of fossil-based oils include montan oil, paraffin oil, microcrystalline oil and intermediate oil. Paraffin oils are mixtures of saturated n- and iso-alkanes, naphthenes, and alkyl- and naphthene-substituted aromatic compounds. Montan oil is a fossilized oil extracted from coal and lignite.

Synthetic oils include oils based on polypropylene, polyethylene, and polytetrafluoroethylene. Other synthetic oils are based on fatty acid amines, Fischer Tropsch, and polyamides, polyethylene and related derivatives. Some oils are obtained by cracking polyethylene at 400° C. The products have the formula (CH₂)ₙH₂, where n ranges between about 50 and 100.

The reader will readily understand that the oil may be an isolated oil or an oil-containing composition, which may include, but without being limited to, a cosmetic carrier (e.g., cream, gel, etc.) containing the oil, an emulsion containing the oil, a plant extract containing the oil, and the like. For example, the oil-containing composition may include commercial products such as any one of VersaPro™ Gel, HRT™ Cream, OleaBase™ Plasticized, PLO Gel Mediflo™, Oral Mix™, and VersaPro™ cream, all from Medisca Pharmaceutique (Canada).

### Cannabis Oil

In some embodiments, the oil-loaded powders of the present disclosure are loaded with a Cannabis oil.

As used herein, the term “Cannabis” generally refers to a genus of flowering plants that includes a number of species. The number of species is currently being disputed. There are three different species that have been recognized, namely Cannabis sativa, Cannabis indica and Cannabis ruderalis. Hemp, or industrial hemp, is a strain of the Cannabis sativa plant species that is grown specifically for the industrial uses of its derived products. A Cannabis oil is typically an oil extracted from Cannabis plant material (e.g., from a Cannabis bud), which can be further processed to purify or isolate one or more ingredients from the Cannabis plant, such as a cannabinoid and/or a terpene.

As used herein, the term “cannabinoid” generally refers to any chemical compound that acts upon a cannabinoid receptor such as CB1 and CB2. Examples of cannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiolic acid (CBDA), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC, defined below), Δ9-tetrahydrocannabinolic acid (THC-A) including either or both isomers 2-COOH-THC (THCA-A) and 4-COOH-THC (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a, 10a-tetrahydrocannabinol (Δ6a, 10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Patent Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-I-yl)-5-pentyl-1,3-benzenediol); Δ4-cannabidiol (2-(6-isopropenyl-3-methyl-4-cyclohexen-I-yl)-5-pentyl-1,3-benzenediol); Δ3-cannabidiol (2-(6-isopropenyl-3-methyl-3-cyclohexen-I-yl)-5-pentyl-1,3-benzenediol); Δ3,7-cannabidiol (2-(6-isopropenyl-3-methylenecyclohex-1-yl)-5-pentyl-1,3-benzenediol); Δ2-cannabidiol (2-(6-isopropenyl-3-methyl-2-cyclohexen-I-yl)-5-pentyl-1,3-benzenediol); Δ1-cannabidiol (2-(6-isopropenyl-3-methyl-I-cyclohexen-I-yl)-5-pentyl-1,3-benzenediol); and Δ6-cannabidiol (2-(6-isopropenyl-3-methyl-6-cyclohexen-I-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ8-tetrahydrocannabivarin (Δ8-THCV), Δ9-cis-tetrahydrocannabinol (cis-THC), Δ9-tetrahydrocannabinol (Δ9-THC), Δ10-tetrahydrocannabinol (Δ10-THC), Δ9-tetrahydrocannabinol-C4 (THC-C4), Δ9 tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

As used herein, the term “terpene” generally refers to refer to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Examples of terpenes known to be extractable from Cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270, which is incorporated herein by reference in its entirety for all purposes.

Cannabis oils have variable viscosity values. Viscosity values for Cannabis oils have been reported in the art, for example: WO2017180660 describes CBD 80%, 60 C: 1240 mPas, CBD 80%, 70 C: 670 mPas, THC 80%, 60 C: 5830 mPas, THC 80%, 70 C: 2200 mPas; Rheosense (Rheometer manufacturers) have an application note on analyzing ‘cannabinoid oils’ where they have measured viscosities at 25 C between 10000 to 80000 mPas using an unspecified shear rate between 60-200 Hz; “Pharmaceutical Formulation Technologies Applicable to Cannabis Product Development” presentation (Vialpando, M., Emerald Conference 2018, available on line), reports a viscosity of THC at 25 C of 100,000 cP.

Processes for extracting Cannabis oils from Cannabis plant material are known in the art. For example, crude Cannabis oils may be obtained from Cannabis plant material using organic solvent extraction, such as extraction with CO₂ (under sub-critical or super-critical conditions), butane, or ethanol. For example, as described in U.S. Pat. No. 6,403,126, the contents of which are incorporated herein by reference in their entirety. Optionally, crude Cannabis oils may be “winterized,” that is, further extracted with ethanol to partially remove Cannabis lipids and oils, as described for example in U.S. Pat. No. 7,700,368, US 2004/0049059, and US 2008/0167483, the contents of each being incorporated herein by reference in their entirety. Thus producing a winterized Cannabis oil. Optionally, winterized Cannabis oils or crude Cannabis oils may be purified with a distillation step to obtain Cannabis oils distillate. US 2016/0346339, which is incorporated herein by reference, describes a Cannabis oil extraction process using solvent extraction followed by filtration, and evaporation of the solvent in a distiller to obtain a distillate. In some embodiments, the cannabinoid content of such Cannabis oils can be of >75% purity as in the case of a winterized Cannabis oil, or >80% purity as in the case of a Cannabis oil distillate.

### Additional One or More Ingredients

In some embodiments, the oil-loaded powders of the present disclosure includes one or more ingredients. For example, an active pharmaceutical ingredient (API), a cosmetic ingredient, a Cannabis-derived ingredient, etc.

Examples of such one or more ingredients include, but are not limited to, antibiotics, analgesics, vaccines, anticonvulsants; anti-asthmatics and vertigo agents, anti-diabetic agents, anti-hypercalcemia agents, anti-fungal agents, anti-neoplastic agents, anti-parkinsonian agents, anti-rheumatic agents, anti-hypertensive, anti-hyperthyroid agent, appetite suppressants, biological response modifiers, cannabinoids, cardiovascular agents, central nervous system stimulants, contraceptive agents, contrast agents, diagnostic agents, dietary supplements (e.g., vitamins, minerals, lipids, saccharides, metals, amino acids (and precursors), nucleic acids and precursors), dopamine receptor agonists, erectile dysfunction agents, fertility agents, gastrointestinal agents, glycerin, hormones, immunomodulators, mast cell stabilizers, muscle relaxants, nutritional agents, opiate antagonist such as trexan (naltrexone HCl), ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, terpenes, T4 and T3 hormone replacement therapy, urinary tract agents, uterine relaxants, vaginal agents, vasodilator, etc.

The one or more ingredients may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base. Non-limiting examples of pharmaceutically acceptable salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate. Another method for defining the ionic salts may be as an acidic functional group, such as a carboxylic acid functional group, and a pharmaceutically acceptable inorganic or organic base. Non-limiting examples of bases include, but are not limited to, hydroxides of alkali metals such as sodium, potassium and lithium; hydroxides of calcium and magnesium; hydroxides of other metals, such as aluminum and zinc; ammonia; and organic amines, such as unsubstituted or hydroxy substituted monotrialkylamines, ditrialkylamines, or trialkylamines; dicyclohexylamine; tributylamine; pyridine; N-methyl-N-ethylamine; diethylamine; triethylamine; mono-, bis- or tris-(2-hydroxy-lower alkyl amines), such as mono-bis-(2-hydroxyethyl)amine, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

### Downstream Applications

In some embodiments, the oil-loaded powder can be used in various industries, including agrochemical, pharmaceutical, medical, food, textile, cosmetic, and hygiene downstream applications. In particular, the oil-loaded powder can be used in Cannabis, pharmaceutical or cosmetic downstream applications.

In some embodiments, the oil-loaded powder can be used for manufacturing precursor products or for manufacturing consumer products. Generally speaking, precursor products are for incorporating (used interchangeably here with infusing, blending, diluting, and the like) a product base so as to obtain a consumer product.

In some embodiments, the consumer products are edible consumer products, such as foodstuffs and beverages. The oil-loaded powder may be combined with any beverage-compatible or food-compatible ingredient. For example, oil-loaded powders of the present disclosure may be used directly in the preparation of foodstuffs and beverages, e.g., as an additive or ingredient. Oil-loaded powders may be used either directly, e.g., as an additive or ingredient, or indirectly e.g., by first dissolving the oil-loaded powder in a solvent (e.g., water) to form a liquid system prior to use. In some embodiments, the oil-loaded powders may be added to beverage or foodstuff directly. In other embodiments, the oil-loaded powders are diluted with a bulking agent. The pre-bulked and/or bulked oil-loaded powders can be packaged for individual servings (e.g., sachets/packets), packages in bulk within a single container, or a combination thereof.

Non-limiting examples of foodstuffs include baked goods (e.g., cookies, brownies, cake, pie, biscuits and pastries), candies (e.g., hard candy, soft candy, gummies, etc.), chocolates, lozenges, gum, mints, dried fruits, nuts, granola, truffles, caramels, chews, taffy, prepared meals, cooking ingredients (e.g., food additives, dry spices, honey, sugar, sweeteners, etc.), ground coffee, instant coffee or tea leaves.

Non-limiting examples of beverage base include water, oil, alcohol; with or without additives or modifiers or both. Such beverage base can be divided into various groups such as plain water, drinking water, milk (both diary and non-diary), juice, a smoothie, coffee or a caffeinated beverage, tea, herbal tea, a cocoa beverage, a carbonated drink, a nitrogenated drink, an energy drink, a drinkable yogurt, a fermented beverage, or an alcoholic or non-alcoholic drink or other hot, room temperature or cold liquids used in drinks. An alcoholic or non-alcoholic drink includes but is not limited to, beer, lager, cider, spirits, wine/fortified wine, and cocktails. Beverages can be caffeinated or non-caffeinated and may contain calories or not. Such beverages may be produced in ready to use form or be produced in a form suitable for preparation in final consumable form at or proximate to the time of ingestion.

In terms of Cannabis applications, a possible downstream application includes manufacturing Cannabis-infused products, e.g., products for recreational and/or medicinal uses. In some embodiments, the Cannabis-infused product is a solid or semi-solid edible product. Edible products come in many forms and can be any product that is suitable, e.g., nontoxic, for placing into the mouth of a human or animal, whether ingested, absorbed, or only chewed or sucked on and at least a portion discarded, etc. In some embodiments, the Cannabis-infused product is a liquid Cannabis-infused product, such as a beverage-non-limiting examples of beverages have been described above. For example, Cannabis-infused liquid composition can be used for ingestion or application to a user's skin or mucous membrane. The liquid compositions may be adapted for topical administration to the skin or mucous membrane. For topical administration, the liquid composition may be prepared as an ointment, tincture, cream, gel, solution including a mouth wash, lotion, spray, aerosol, dry powder for inhalation, suspension, and the like. A preparation for topical administration to the skin can be prepared by mixing the liquid composition with non-toxic, therapeutically inert, solid or liquid carriers customarily used in such preparations. Examples of Cannabis-infused liquid compositions are described in US 2021/0298340, the contents of which are hereby incorporated by reference in their entirety.

Cannabis oil-loaded powders have been reported to have reduced bitter-tasting characteristics, which can be suitable for making Cannabis-infused products (e.g., see WO 2022/140849). In contrast to common approaches that aim to mask bitter and off-taste in Cannabis-infused product with the addition of masking ingredients or flavors (e.g., sugars), the herein described Cannabis oil-loaded powders can effectively mask the bitter and off-taste of Cannabis-infused products while avoiding having to add such taste masking ingredients or flavors.

In some embodiments, the Cannabis oil-loaded powder can be used by its manufacturer to obtain the Cannabis-infused product, ready for packaging and commercialization. In some embodiments, the Cannabis oil-loaded powder may be packaged and sold to a product manufacturer, which can then use same to obtain the Cannabis-infused product. In some embodiments, the Cannabis oil-loaded powder may be packaged and commercialized alone or together with a base product such that the end-user may use same to obtain the Cannabis-infused product.

In terms of pharmaceutical applications, a possible downstream application includes incorporating the oil-loaded powders into compressed solid dosage forms, such as tablets and caplets (i.e., capsule-shaped tablets). There are three well known processes for manufacturing compressed solid dosage forms: the wet granulation method, the double-compression method (also known as dry granulation) and the direct compression method.

In the wet granulation process, a pre-measured quantity of the drug and one or more additional components, such as a diluent, are combined. This mixture is then thoroughly mixed with a liquid, often water or ethanol, resulting in the particles coming together to form a damp mass. Sometimes, a binder is included in the liquid. The damp mass is screened to create granules, which are subsequently dried. These dry granules are sieved to achieve a specific size, and then they are typically mixed with a solid lubricant and, potentially, other components. Finally, these lubricated granules, along with any extra-granular ingredients, are compressed to form a tablet, which may undergo a coating process.

In contrast, the double-compression or dry granulation method is a more streamlined process with fewer stages than wet granulation. It does not involve contact with a liquid or drying, making it particularly suitable for drugs sensitive to water and heat. In this method, the drug and other ingredients, such as a lubricant, are blended and then subjected to an initial compression step. There are two common techniques for this first compression: roller compaction, where the blend is passed between rollers to create sheets, and slugging, where the blend is compressed into larger tablet-like forms known as slugs. These resulting sheets or slugs are subsequently broken down into granules, mixed with a solid lubricant, and then compressed in a second step to produce the final tablet.

The direct compression method is the simplest among the three well-established techniques for manufacturing solid compressed dosage forms. In this approach, the drug and any other required components are mixed together, and directly compressed into the final solid compressed dosage form. However, it is crucial that the ingredients have good flow characteristics and exhibit cohesion to be suitable for direct compression tableting. Commonly used diluents in direct compression tableting include microcrystalline cellulose and lactose.

In terms of cosmetic applications, a possible downstream application includes incorporating the oil-loaded powders into creams, lotions, gels, ointments, and the like.

For example, pharmaceutical or cosmetic activities can be performed by licensed operators (e.g., pharmacist, 503B outsourcing compounding facility, medical doctor, pharmaceutical manufacturer, cosmetic manufacturer, etc.).

Without being limited by any theory, the present inventor believes that the herein described oil-loaded powders can facilitate the dispensing, measuring, or dosing of specific amounts of oil using conventional means in comparison to doing so with the oil per se. Indeed, it can be more difficult to handle oils due to the increased viscosity and/or stickiness in comparison to oil-loaded powders, which are substantially dry and free flowing. It is also easier to distribute the oil-loaded powders throughout whole batches homogenously during the manufacturing process by conventional mixing operations compared to oil per se.

### EXAMPLES

Details of specific practical implementation of the present disclosure will be further described in the following non-limiting examples.

### Example 1

In this example, ingredients were placed in a container, and the ingredients were mixed by imparting movements to the container in order to obtain an oil-loaded powder.

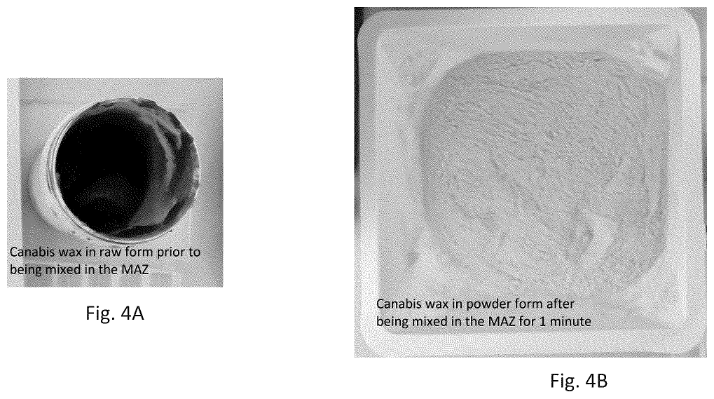

In a first step, about 20 g of Cannabis crude oil/wax (containing CBD) and 10 g of carrier particles were placed in a 310 ml High-density polyethylene (HDPE) plastic container, as shown in FIG. 4A. The carrier particles in this case were a mixture of microcrystalline cellulose, silicon dioxide (silica), and hydrated colloidal silica. The oil/carrier particles ratio (w/w) being of 2:1.

In a second step, the HDPE container was placed in a container holder of a MAZ™ KK-300 mixer and the ingredients were mixed by imparting superimposed revolution and rotation movements to the container. The MAZ KK-300 mixer is a bladeless mixer. The mixing was performed for 20 seconds at 2000 rpm, and then for 1 minute at 900 rpm, resulting in a free flowing Cannabis oil-loaded powder as shown in FIG. 4B.

### Example 2

In this example, ingredients were placed in a container, and the ingredients were mixed by imparting movements to the container in order to obtain an oil-loaded powder.

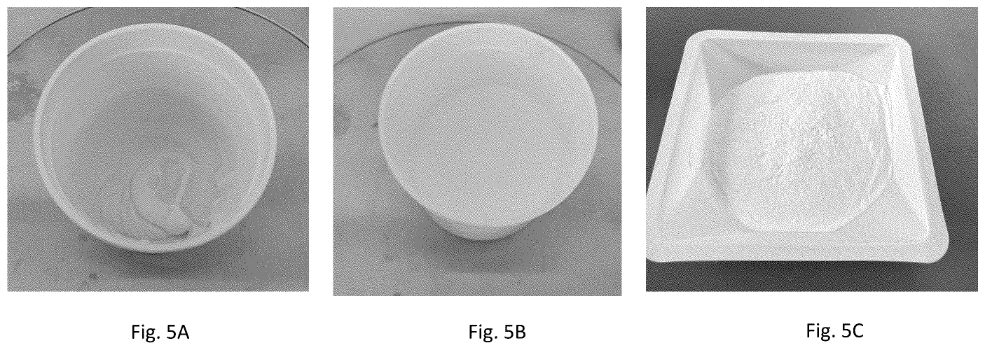

In a first step, about 20 g of an oil-containing composition (i.e., oil-in-water emulsion Versapro™ Cream base, Medisca Pharmaceutique Inc. Canada) and 10 g of carrier particles were placed in a 310 ml HDPE plastic container, as shown in FIG. 5A. The carrier particles in this case were a mixture of microcrystalline cellulose, silicon dioxide (silica), and hydrated colloidal silica. The oil/carrier particles ratio (w/w) being of 2:1.

In a second step, the HDPE container was placed in a container holder of a MAZ™ KK-300 mixer and the ingredients were mixed by imparting superimposed revolution and rotation movements to the container. The mixing was performed for 20 seconds at 2000 rpm, and then for 1 minute at 900 rpm, resulting in a free flowing oil containing composition-loaded powder as shown in FIG. 5C.

### Example 3

In this example, ingredients were placed in a container, and the ingredients were mixed by imparting movements to the container in order to obtain an oil-loaded powder with aggregates.

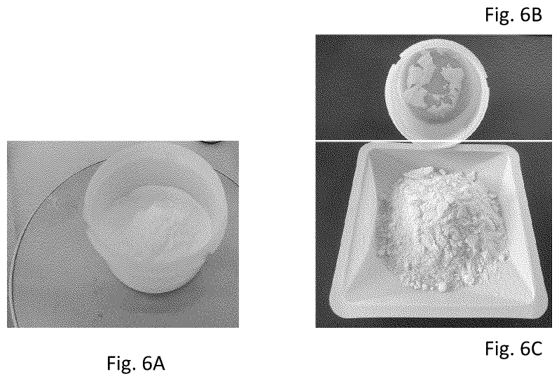

In a first step, about 8 g of the oil-containing composition of Example 2 and about 27 g of carrier particles were placed in a 310 ml HDPE plastic container, as shown in FIG. 6A. The carrier particles in this case were a mixture of microcrystalline cellulose, silicon dioxide (silica), and hydrated colloidal silica. The oil/carrier particles ratio (w/w) being of 1:3.

In a second step, the HDPE container was placed in a container holder of a MAZ KK-300 mixer and the ingredients were mixed by imparting superimposed revolution and rotation movements to the container. The MAZ KK-300 mixer is a bladeless mixer. The mixing was performed for 1 minute at 2000 rpm, resulting in a portion of the cream that was still humid and stuck to the bottom of the container as shown in FIG. 6B, and a portion that was a clumpy and non-uniform oil composition-loaded powder as shown in FIG. 6C.

### Example 4

In this example, ingredients were placed in a container, and the ingredients were mixed by imparting movements to the container in order to obtain an oil-loaded powder, which was then tested for oil distribution in the powder blend.

In a first step, about 33.33 g of full spectrum Cannabis extract and about 16.67 g of carrier particles were placed in a 310 ml HDPE plastic container. The carrier particles in this case were NanoSil™ particles (Pete Pharmaceuticals, USA). The oil/carrier particles ratio (w/w) being of 2:1.

In a second step, the HDPE container was placed in a container holder of a MAZ™ KK-400WH mixer and the ingredients were mixed by imparting superimposed revolution and rotation movements to the container. The MAZ KK-400WH mixer is a bladeless mixer. The mixing was performed for 120 seconds at 2000 rpm resulting in a free flowing oil-loaded powder.

The volume of the oil-loaded powder was then divided into three equal divisions across the vertical axis of the container: top, middle, and bottom layers. This process was repeated for two sample sets, for a total of 6 assay sample jars. Subsequently, the samples were sent for HPLC testing, whereby the concentrations of each of the constituent ingredient within each sample jar were determined.

HPLC testing was performed for all samples, labelled Mix 1 and Mix 2, found in the table 2 below, to determine the concentration of full spectrum Cannabis extract. In addition, two trials were performed for each sample and a third trial was calculated using the average from trial 1 and 2. Furthermore, the final average of full spectrum Cannabis extract and relative standard deviation (RSD) was also calculated. Results can be found in table 2 below.

TABLE 2
HPLC Testing Results

| First Trial Analysis | Mix 1 | Mix 2 |
|---|---|---|
| Top (CBD Assay) | .665 | .687 |
| Middle (CBD Assay) | .663 | .651 |
| Bottom (CBD Assay) | .661 | .677 |
| Duplicate Trial Analysis | Mix 1 | Mix 2 |
| Top (CBD Assay) | .668 | .696 |
| Middle (CBD Assay) | .658 | .647 |
| Bottom (CBD Assay) | .678 | 655 |
| Average of Duplicate Analysis | Mix 1 | Mix 2 |
| Top (CBD Assay) | .667 | .692 |
| Middle (CBD Assay) | .660 | .649 |
| Bottom (CBD Assay) | .670 | .666 |
| | Mix 1 | Mix 2 |
| Average CBD Assay | .665 | .668 |
| Relative Standard Deviation CBD Assay | 0.08335 | 0.08332 |

This test thus showed that a substantially homogenous oil distribution was achieved throughout the particles of the oil-loaded powder—i.e., indeed, the relative standard deviation (RSD) obtained here of ≤0.8% is considered indicative of the powder blend having substantially homogenous oil distribution.

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art considering the present description that numerous modifications and variations can be made. The scope of the invention is defined more particularly in the appended claims.

1. A method for manufacturing an oil-loaded powder, the method comprising
placing ingredients in a container body of a container; and
mixing the ingredients to produce the oil-loaded powder with a mixer imparting superimposed rotation and revolution movements to the container containing the ingredients,
wherein the ingredients comprise a carrier in particulate form and an oil, wherein the carrier is configured for oil sorption.

2. The method of claim 1, wherein the mixer is a planetary mixer.

3. The method of claim 1, wherein the mixer is a bladeless mixer.

4. The method of claim 1, wherein the carrier includes micron size particles having a surface area or porous structure capable of oil sorption.

5. The method of claim 4, wherein the carrier is a mesoporous particle.

6. The method of claim 4, wherein the carrier includes microcrystalline cellulose, silicon dioxide, hydrated colloidal silica, or any combination thereof.

7. The method of claim 4, wherein the carrier includes a mesoporous internal network for sorption of the oil.

8. The method of claim 1, wherein the mixing includes selecting and/or obtaining a mixing parameter.

9. The method of claim 8, wherein the mixing parameter includes mixing time, mixing speed, maximal g force, container size, ingredients amounts, or a combination thereof.

10. The method of claim 1, wherein the oil-loaded powder is characterized with a substantially uniform oil distribution and/or substantially uniform mean particle size.

11. The method of claim 1, wherein the oil is a plant-based, animal-based, fossil-based, or synthetic oil.

12. The method of claim 11, wherein the plant-based oil is a Cannabis oil.

13. The method of claim 1, wherein the ingredients have a ratio oil/carrier particles (w/w) of from about 5:1 to about 1:1.

14-26. (canceled)

27. A method for manufacturing a consumer product containing an oil-loaded powder, the method comprising:
placing a carrier in particulate form configured for oil sorption and an oil in a container body of a container;
mixing the carrier and the oil to produce the oil-loaded powder with a mixer configured for imparting superimposed rotation and revolution movements to the container containing the ingredients; and
incorporating the oil-loaded powder in a base product to produce the consumer product.

28. The method of claim 27, wherein the consumer product includes an edible consumer product.

29. The method of claim 27, wherein the consumer product includes a pharmaceutical product.

30. The method of claim 27, wherein the consumer product includes a cosmetic product.

31. An oil-loaded powder comprising carrier particles loaded with the oil by subjecting the powder and oil to superimposed rotation and revolution movements, wherein the powder has a substantially uniform distribution of the oil throughout the particles.

32. The oil-loaded powder of claim 31, wherein the carrier particles have a particle size distribution (PSD) of between about 1 μm and about 1000 μm.

33. The oil-loaded powder of claim 31, wherein the particles have a specific surface area of between about 25 m²/g to about 1000 m²/g.

34. The oil-loaded powder of claim 31, wherein the particles have an oil sorption capacity of from about 2.0 ml/g to about 4.0 ml/g.

35. The oil-loaded powder of claim 31, wherein the powder exhibits an oil concentration gradient having≤ 3% relative standard deviation (% RSD), when measured at least at the top, middle and bottom layers of the oil-loaded powder.

36. The oil-loaded powder of claim 31, wherein the powder has an angle of repose of below 40°.

37. The oil-loaded powder of claim 31, wherein the oil is a plant-based, animal-based, fossil-based, or synthetic oil.

38. The oil-loaded powder of claim 37, wherein the plant-based oil is a Cannabis oil.

39. A consumer product comprising an oil-loaded powder, wherein the oil-loaded powder includes carrier particles loaded with the oil by the method according to claim 27, wherein the powder has a substantially uniform distribution of the oil throughout the particles.

40. The consumer product of claim 39, wherein the consumer product includes an edible consumer product.

41. The consumer product of claim 39, wherein the consumer product includes a pharmaceutical product.

42. The consumer product of claim 39, wherein the consumer product includes a cosmetic product.
